(19) United States
(12) Patent Application Publication
(10) Pub. No.: US 2002/0106670 A1
(43) Pub. Date: Aug. 8, 2002

(54) Methods for identifying novel therapeutic agents

(76) Inventors: John L. Herrmann, Guilford, CT (US); Lucas Rastelli, Guilford, CT (US); Catherine E. Burgess, Wethersfield, CT (US); Bonnie E. Gould-Rothberg, Guilford, CT (US); Jonathan M. Rothberg, Guilford, CT (US); Richard A. Shimkets, West Haven, CT (US)

Correspondence Address: Ivor R. Elrifi, Ph.D., MINTZ. LEVIN, COHN, FERRIS, GLOVSKY and POPEO, P.C., One Financial Center, Boston, MA, 02111, US

(21) Appl. No.: 09/945,980

(22) Filed: Sep. 4, 2001

Related U.S. Application Data
(60) Provisional application No. 60/229,847, filed on Sep. 1, 2000.

(51) Int. Cl. C12Q 1/68; G06F 19/00; G01N 33/48; G01N 33/50

(52) U.S. Cl. 435/6; 702/20

(57) ABSTRACT

The invention provides a method for identifying a therapeutic agent. The method includes detecting a nucleic acid in a test sample, e.g. cells, cell lines or tissue, which contains a plurality of nucleic acid species, determining if the detected nucleic acid contributes to a disease state and is thus a qualified therapeutic target, and establishing if the qualified therapeutic target plays a role in disease progress and is thus a verified therapeutic candidate that can function as a therapeutic agent.

### RELATED APPLICATIONS

This application claims priority from U.S. Ser. No. 60/229,847 filed Sep. 1, 2000, which is incorporated by reference in its entirety.

### BACKGROUND OF THE INVENTION

A “new biology” is poised to deliver improved therapeutics that target specific molecular alterations that contribute to the development and progression of human malignancies. Many of these drugs target specific regulatory factors that are well established for their respective roles in tumor invasion and metastasis, angiogenesis, cell cycle, and resistance to therapy. For the most part these targets have been discovered by model-driven experimental studies based on laboratory and clinical observations.

Perhaps the latest of the new biologies that is poised to deliver new “druggable” targets for human disease is the field of study called “functional genomics”. This field employs a new approach that is poised to revolutionize various aspects of cancer research and the practice of oncology. Functional genomics is anticipated to bring about a sizeable advance in how new anticancer therapeutics are discovered and developed as well as how cancer is detected and classified resulting in more tailored therapies.

The explosion of information generated by large-scale functional genomics technologies has resulted in an exponential increase in the number of potential genes and proteins available for pharmaceutical and diagnostic research development. In order to tap this potential, a primary challenge is to develop a strategy to effectively integrate and extract meaning from human genomic sequence information.

### SUMMARY OF THE INVENTION

The invention is based in part on a discovery of a method for identifying a nucleic acid from a sample containing a plurality of nucleic acid species, determining its expression in various disease states and establishing its utility as a therapeutic agent. The invention can be carried out using a series of experimental methods.

In one aspect, the invention provides a method for identifying a therapeutic agent. The method includes detecting a nucleic acid in a test sample, e.g. cells, cell lines or tissue, which contains a plurality of nucleic acid species, determining if the detected nucleic acid contributes to a disease state and is thus a qualified therapeutic target, and establishing if the qualified therapeutic target plays a role in disease progress and is thus a verified therapeutic candidate that can function as a therapeutic agent.

In some embodiments, the nucleic acids, e.g. mRNA or cDNA molecules, are detected using differential gene expression, where the expressed genes in the test sample are compared to those genes expressed in a reference sample.

In other embodiments, detection of nucleic acids with differential gene expression is accomplished by: (a) probing the sample with one or more recognition means, each recognition means recognizing a different target nucleotide subsequence or a different set of target nucleotide subsequences; (b) generating one or more output signals from the sample probed by the recognition means, each output signal being produced from a nucleic acid in the sample by recognition of one or more target nucleotide subsequences in the nucleic acid by the recognition means and comprising a representation of (i) the length between occurrences of target nucleotide subsequences in the nucleic acid, and (ii) the identities of the target nucleotide subsequences in the nucleic acid or the identities of the sets of target nucleotide subsequences among which are included the target nucleotide subsequences in the nucleic acid; and (c) searching a nucleotide sequence database to determine sequences that are predicted to produce or the absence of any sequences that are predicted to produce the one or more output signals produced by the nucleic acid acid, the database comprising a plurality of known nucleotide sequences of nucleic acids that may be present in the sample, a sequence from the database being predicted to produce the one or more output signals when the sequence from the database has both (i) the same length between occurrences of target nucleotide subsequences as is represented by the one or more output signals, and (ii) the same target nucleotide subsequences as are represented by the one or more output signals, or target nucleotide subsequences that are members of the same sets of target nucleotide subsequences represented by the one or more output signals.

In another embodiment, the method includes providing a population of nucleic acid sequences; partitioning said population into one or more subpopulations of nucleic acids; identifying a first nucleic acid sequence in the subpopulation of nucleic acid sequences; and comparing the first nucleic acid sequence to a reference nucleic acid sequence or sequences, wherein the absence of the first nucleic acid sequence in the reference nucleic acid or nucleic acid sequences indicates the first nucleic acid is a novel nucleic acid sequence.

In some embodiments, detected nucleic acids are determined to be qualified therapeutic targets using several methods, including but not limited to; laser capture microdissection, serial analysis of gene expression (SAGE), detection of protein-protein interactions involving the protein encoded by the identified nucleic acid or real time quantitative polymerase chain reaction carried out on a plurality of test samples. This embodiment can also include a combination of any two or more of these methodologies.

In some embodiments, qualified therapeutic targets are established as verified therapeutic targets, and thus therapeutic agents, by demonstrating the targets ability to inhibit gene expression by utilizing antisense nucleic acids, by utilizing an associated antibody to modulate a function of a protein or polypeptide encoded by a detected nucleic acid or by using associated chemical compounds to modulate a function of a protein or polypeptide encoded by a detected nucleic acid. Further methods include transforming a cell with a detected nucleic acid to assess the function of a protein or polypeptide encoded by a detected nucleic acid or by utilizing a mammal harboring a transgene of a detected nucleic acid to assess the function of a protein or polypeptide encoded by a detected nucleic acid. This embodiment can also include a combination of any two or more of these methods.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is an overview of the process of genomics-based oncologic drug target discovery.

FIG. 2 shows the expression profile of a novel, potentially secreted, protein likely to have a characteristic enzymatic activity, (2A and 2C) compared with the profile of Her-2 (2B and 2D). Expression profiling was accomplished using quantitative real-time PCR on panels of RNA isolated from tumor derived cell lines and human normal tissues (2A and 2B) and tumor tissues many having a match from the surgical margin for comparisons (2C and 2D). In the last panel, the tumor derived from the same tissue are grouped and color coded together with the corresponding normal tissue.

### DETAILED DESCRIPTION OF THE INVENTION

Therapeutically relevant targets can be approached using the herein described methods. In general, a nucleic acid is detected and a determination is made that the nucleic acid points to a qualified therapeutic agent. Next, an assessment is made that the qualified target points to a validated therapeutic agent. Validated therapeutic agents are considered to be new therapeutic agents for an indicated disease. The rationale and general strategy for these approaches are discussed in the sections that follow.

Detecting Nucleic Acids in Test Sample

A nucleic acid is first identified in a sample as being associated with a particular diseased state. The nucleic acid is taken from a cell or tissue population for which the diseased state is known. In some embodiments, comparison of the gene expression profile in the test cell population to the reference cell population reveals the presence, or degree, of the measured parameter depends on the composition of the reference cell population.

If desired, comparison of differentially expressed sequences between a test cell population and a reference cell population can be done with respect to a control nucleic acid whose expression is independent of the parameter or condition being measured. Expression levels of the control nucleic acid in the test and reference nucleic acid can be used to normalize signal levels in the compared populations.

In some embodiments, the test cell population is compared to multiple reference cell populations. Each of the multiple reference populations may differ in the known parameter, or disease state. The test cell population can be any number of cells, i.e., one or more cells, and can be provided in vitro, in vivo, or ex vivo.

In other embodiments, the test cell population can be divided into two or more subpopulations. The subpopulations can be created by dividing the first population of cells to create as identical a subpopulation as possible. This will be suitable, in, for example, in vitro or ex vivo screening methods. In some embodiments, various sub populations can be exposed to a control agent, and/or a test agent, multiple test agents, or, e.g., varying dosages of one or multiple test agents administered together, or in various combinations.

Preferably, cells in the reference cell population are derived from a tissue type as similar as possible to test cell. For example, the reference cell population can be a database of expression patterns from previously tested cells for which one of the herein-described parameters or conditions. The association can be based on, e.g., correlation of levels of a transcript of a gene and the presence of a diseased state, or of particular forms of a nucleic acid sequence (e.g., a particular form of a gene) and the diseased state.

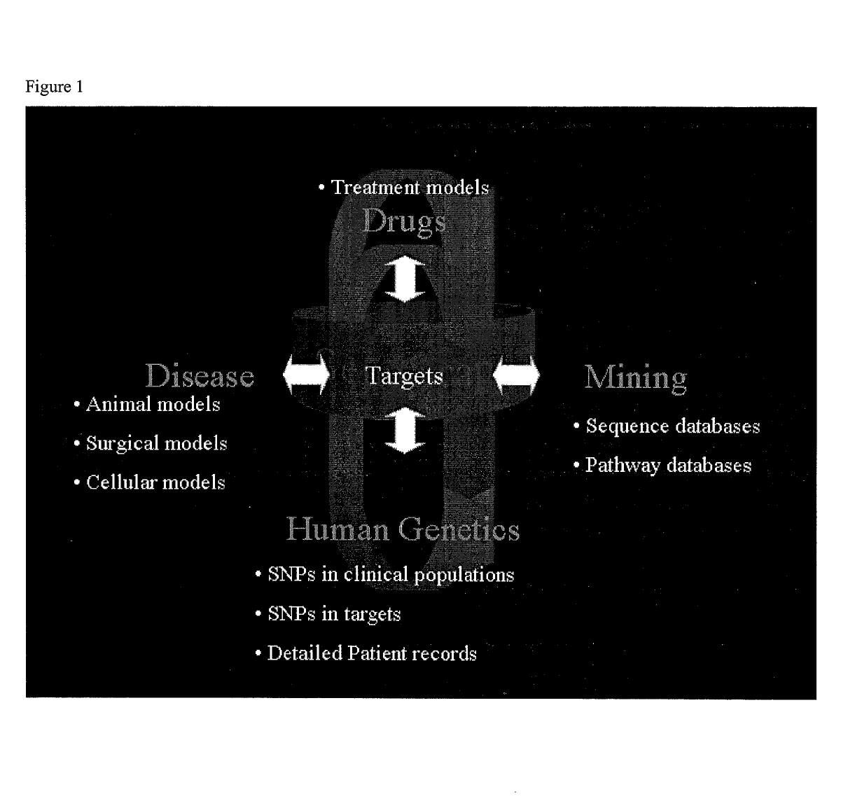

The initial association can be made with several methods recognized in the art for detecting nucleic acids in a test sample. Some of these methods are indicated schematically in FIG. 1. These approaches including mining the genome for novel sequences and novel biological pathways, gene expression analysis in studies based on medical and experimental hypotheses using disease models, and use of human genetics studies to identify genetic factors associated with cancer using SNP. Targets can then be qualified and validated using the same approaches.

A preferred method for detecting the association of a particular nucleic acid and gene is with the methods and apparatuses is differential gene expression. Many methods of differential gene expression are known in the art. One method, termed differential display, is described in Liang and Pardee, Science 257:967-71, 1992. Differential display is a transcript amplification and imaging technology for detection of changes in gene expression in a comparison of multiple experimental samples. This method has been used: 1) to identify a ribonucleotide reductase gene involved p53-dependent cell-cycle checkpoint control following genotoxic stress (Tanaka et al., Nature 404:42-49, 2000); 2) to identify a proliferation-associated SNF2-like gene (PASG) altered in leukemia (Lee et al., Cancer Research 60:36123622, 2000); and 3) to link gene expression patterns to therapeutic groups in breast cancer potentially offering the opportunity for fine tuned prognostic accuracy and tailored therapy (Martin et al., Cancer Research 60:2232-2238, 2000). Differential display allows for the systematic visualization of the repertoire of expressed genes from different experimental samples in simple side-by-side comparisons.

Alternatively, nucleic acids can be detected using gene microarray hybridization. Microarray technology allows for profiling of gene expression on a large scale by means of miniaturized, high-density arrays of oligonucleotide probes tethered to a solid support or “chip”. These probes correspond to full-length genes as well as uncharacterized expressed sequence tags (ESTs). Once fabricated, the cDNA microarray chips are hybridized to RNA isolated from an experimental sample that has been amplified and labeled with a fluorescent reporter group. After the hybridization reaction is complete, the array is scanned to generate a map of the patterns of hybridization. The hybridization data are collected as light emitted from the fluorescent reporter groups incorporated into the labeled target bound to the probe array. Probes that most significantly match the target generally produce stronger signals than those with significant mismatches. Since the sequence and position of each probe on the array are known, by complementarity, the identity of the target transcript applied to the microarray can be predicted. The main difference between this technology and those previously described, is the limitation of analyzing only those sequences present on the microarray.

For experimental studies involving cDNA microarrays, clustering algorithms have been developed to aid in the deconvolution of these extensive gene expression data sets. One such study that highlights the impact of this technology on genomics-based drug target is the evaluation of quiescent human fibroblasts. The study provided an analysis of the global alterations in the gene expression of quiescent fibroblasts stimulated to proliferate by the addition of serum (Iyer et al., Science 283:83-87, 1999). Microarray hybridization has also been used to distinguish between two distinct forms of diffuse large B-cell lymphoma. Variations have been identified based on tumor proliferation rate, host response and the differentiation state of the tumor (Alidade et al., Nature 403:503-511, 2000).

Another type of differential gene expression is described in, e.g., U.S. Pat. No. 5,871,697 and in Shimkets et al., Nat. Biotech. 17:798-803, 1999. Biologically derived DNA sequences in a mixed sample or in an arrayed single sequence clone can be determined and classified without sequencing in a process known as GENECALLING® analysis. The mRNA profiling technique for determining differential gene expression utilizes, but does not require, prior knowledge of gene sequences. This method permits high-throughput reproducible detection of most expressed sequences with a sensitivity of greater than 1 part in 100,000. Gene identification by database query of a restriction endonuclease fingerprint, confirmed by competitive PCR using gene-specific oligonucleotides, facilitates gene discovery by minimizing isolation procedures.

The methods make use of information on the presence of carefully chosen target subsequences, typically of length from 4 to 8 base pairs, and preferably the length between target subsequences in a sample DNA sequence together with DNA sequence databases containing lists of sequences likely to be present in the sample to determine a sample sequence. One preferred method uses restriction endonucleases to recognize target subsequences and cut the sample sequence. Carefully chosen recognition moieties are ligated to the cut fragments, the fragments amplified, and the experimental observation made. Polymerase chain reaction (PCR) is a preferred method of amplification. Alternatively, information on the presence or absence of carefully chosen target subsequences in a single sequence clone together with DNA sequence databases are used to determine the clone sequence. Computer implemented methods can be used analyze the experimental results and to determine the sample sequences in question and to carefully choose target subsequences in order that experiments yield a maximum amount of information.

Preferably, sequences are further analyzed using methods described in, e.g., U.S. Pat. No. 6,190,868 and Shimkets et al., Nat. Biotech. 17:798-803, 1999. The methods provide positive confirmation that nucleic acids, possessing putatively identified sequence predicted to generate observed GENECALLING® signals, are actually present within the sample from which the signal was originally derived. The putatively identified nucleic acid fragment within the sample possesses 3′- and 5′-ends with known terminal subsequences, the method comprising; contacting the nucleic acid fragments in the sample in amplifying conditions with (i) a nucleic acid polymerase; (ii) “regular” primer oligonucleotides having sequences comprising hybridizable portions of the known terminal subsequences; and (iii) a “poisoning” oligonucleotide primer, said poisoning primer having a sequence comprising a first subsequence that is a portion of the sequence of one of said known terminal subsequences and a second subsequence that is a hybridizable portion of said putatively unidentified sequence which is adjacent to said one known terminal subsequence, wherein nucleic acids amplified with said poisoning primer are distinguishable upon detection from nucleic acids amplified with said nucleic acids amplified only with said regular primers; separating the products of the contacting step; and the detecting sequence is confirmed if the nucleic acids amplified with said poisoning primer are detected.

Nucleic acids can also be identified using methods disclosed in WO00/40757. Nucleic acids in a sample of nucleic acids can be identified in which nucleic acids are initially present in unequal amounts. The starting population of nucleic acids are partitioned to form one or more subpopulations, and nucleic acids that are present in different amounts in the partitioned nucleic acid sample as compared to the starting population are identified.

Differential gene expression can also be assessed using the Serial Analysis of Gene Expression or SAGE (Velculescu et al., Science 270:484-487, 1995). SAGE can also be adapted to high-throughput approaches to differential gene expression analysis but differs considerably in its core method. Unlike transcript amplification and imaging, SAGE does not directly quantify the expression level of a gene, but rather it scores “tags” which are digital representations of the mRNA product(s) of a gene. A SAGE “tag” is a nucleotide sequence of a defined length, directly 3′-adjacent to the 3′-most restriction site for a particular restriction enzyme. SAGE technology has been used to prepare an evaluation of gene expression profiles in gastrointestinal tumors (Zhang et al., Science 276:1268-1272, 1997); the delineation of transcriptional targets of p53 that modulate p53-dependent apoptosis (Polyak et al., Nature 389:300-305, 1997); and the identification of myc as a downstream target of the APC tumor suppressor gene (He et al., Science 281:1509-1512, 1998).

Determining that Detected Nucleic Acids are Associated with Qualified Therapeutic Candidates

A detected nucleic acid is then subject to further analysis to determine whether it is associated, or points to, a qualified therapeutic candidate. One approach to deal with the enormous complexity in tissue heterogeneity relies on the differential gene expression techniques mentioned above.

A second method uses laser-capture microdissection, or LCMD, to tease apart the tissues to be analyzed. The analysis of gene expression patterns is then focused on comparing similar components in malignancies and normal tissues (Emmert-Buck et al., Science 274:998-100, 1996). LCMD permits the investigator to isolate single cells and groups of cells representing various subpopulations of interest within a tumor. The resulting 2D map of gene expression data overlayed with histopathological information can be further enhanced with regard to usefulness by a third layer of patient longitudinal data providing a three-dimensional model of cancer.

Determination of a qualified therapeutic candidate can also be determined using protein-protein interaction. One way to characterize the function of a protein is to identify other proteins with known function that bind to it thereby inferring function upon the uncharacterized protein. Methods for detecting protein-protein interactions are described in, e.g., U.S. Pat. No. 6, 083,693 and Uetz et al., Curr Opin. Microbiol 3:303-8, 2000).

These references describe methods for detecting protein—protein interactions, among two populations of proteins, each having a complexity of at least 1,000. For example, proteins are fused either to the DNA-binding domain of a transcriptional activator or to the activation domain of a transcriptional activator. Two yeast strains, of the opposite mating type and carrying one type each of the fusion proteins are mated together. Productive interactions between the two halves due to protein—protein interactions lead to the reconstitution of the transcriptional activator, which in turn leads to the activation of a reporter gene containing a binding site for the DNA-binding domain. This analysis can be carried out for two or more populations of proteins. The differences in the genes encoding the proteins involved in the protein—protein interactions are characterized, thus leading to the identification of specific protein—protein interactions, and the genes encoding the interacting proteins, relevant to a particular tissue, stage or disease. Furthermore, inhibitors that interfere with these protein-protein interactions are identified by their ability to inactivate a reporter gene. The screening for such inhibitors can be in a multiplexed format where a set of inhibitors will be screened against a library of interactors.

Resources cataloging protein-protein interactions are also described atv KEGG (<http://www.genome.ad.jp/>) maintained by the Institute for Chemical Research, Kyoto University and CSNDB, the Cell Signaling Networks DataBase (<http://geo.nihs.go.jp/csndb/>) maintained by the National Institute of Health Sciences.

For a database of selected novel genes, homology information is preferably integrated with expression analysis to determine both the normal tissue distribution and to define any potential disease correlation(s). One approach to accomplish this objective is to analyze transcript abundance for each novel gene across hundreds or thousands of human cell lines and tissue specimens (diseased and matched normal) using a technology such as quantitative real-time PCR. Preferably, a relatively restricted normal tissue distribution which affords a good therapeutic window coupled with a strong, statistically significant dysregulation in human malignancy is obtained. Although not necessary, the drug target discovery process is accelerated if the expression patterns also reveals the gene of interest to be dysregulated in one or more cancer cell lines that can be grown as tumor xenografts in nude mice. These novel sequences may then be evaluated using any number of target validation approaches.

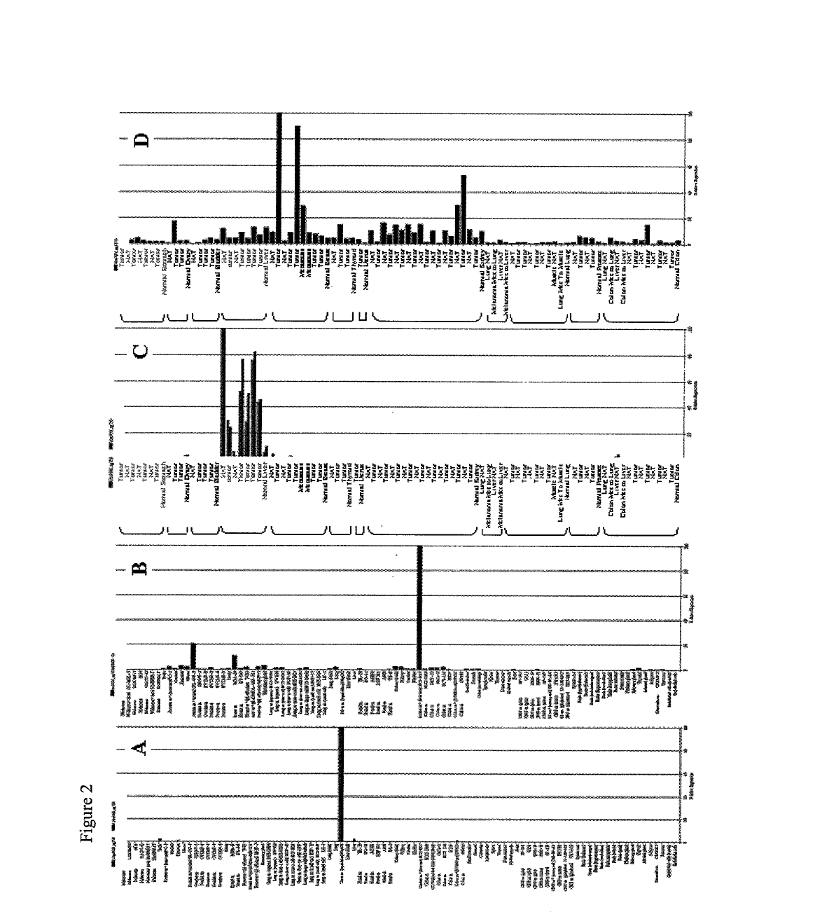

An example of the application of mining strategies to discern potential therapeutic targets is highlighted in FIG. 2. Shown is the expression profile of an identified novel gene. The expression profile reveals a good therapeutic window, being expressed only by hepatoma cell line and hepatocellular carcinomas. Homology analysis reveals that this gene may have a characteristic enzymatic activity. This protein is likely to be secreted making it a potential small molecule drug target or an antibody target. This expression profile is compared with that of Her-2, the target of Herceptin for the treatment of breast cancer. The comparison suggests that a therapeutic antibody directed against this protein will have a very good potential to treat liver cancer.

Establishing a Validated Therapeutic Candidate

Validation studies to establish a target “qualified” target by virtue of disease association. Validation demonstrates that the target actually contributes to disease development and progression, or occurs as a consequence of disease progression. Validation can be established using any technology known in the art. Preferred methods include antisense, antibody, cellular transformation, and studies with transgenic animals.

### Equivalents

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications are considered to be within the scope of the following claims.

What is claimed is:

1. A method of identifying a therapeutic agent comprising the steps of:
a) detecting a nucleic acid in a test sample wherein said test sample comprises a plurality of nucleic acid species;
b) determining that said detected nucleic acid is associated with a qualified therapeutic candidate; and
c) establishing that said qualified therapeutic candidate is a validated therapeutic candidate; whereby said verified therapeutic candidate is a therapeutic agent.

2. The method of claim 1 wherein said nucleic acid species are mRNA molecules.

3. The method of claim 1 wherein said nucleic acid species are cDNA molecules.

4. The method of claim 1 wherein said detecting step comprises differential gene expression, and wherein said differential gene expression compares the expression of genes between a test state and a reference state different from said test state.

5. The method of claim 4 wherein said differential gene expression comprises
(a) probing said sample with one or more recognition means, each recognition means recognizing a different target nucleotide subsequence or a different set of target nucleotide subsequences;
(b) generating one or more output signals from said sample probed by said recognition means, each output signal being produced from a nucleic acid in said sample by recognition of one or more target nucleotide subsequences in said nucleic acid by said recognition means and comprising a representation of (i) the length between occurrences of target nucleotide subsequences in said nucleic acid, and (ii) the identities of said target nucleotide subsequences in said nucleic acid or the identities of said sets of target nucleotide subsequences among which are included the target nucleotide subsequences in said nucleic acid; and
(c) searching a nucleotide sequence database to determine sequences that are predicted to produce or the absence of any sequences that are predicted to produce said one or more output signals produced by said nucleic acid, said database comprising a plurality of known nucleotide sequences of nucleic acids that may be present in the sample, a sequence from said database being predicted to produce said one or more output signals when the sequence from said database has both (i) the same length between occurrences of target nucleotide subsequences as is represented by said one or more output signals, and (ii) the same target nucleotide subsequences as are represented by said one or more output signals, or target nucleotide subsequences that are members of the same sets of target nucleotide subsequences represented by said one or more output signals.

6. The method of claim 1 wherein said determining step comprises
a) laser capture microdissection,
b) serial analysis of gene expression (SAGE),
c) detection of protein-protein interactions wherein at least one of the proteins is a polypeptide encoded by a detected nucleic acid, or
d) real time quantitative polymerase chain reaction carried out on a plurality of samples drawn from various cells, cell lines or tissues, or a combination of any two or more of said determinations.

7. The method of claim 1 wherein said establishing step comprises
a) inhibiting gene expression by application of an antisense nucleic acid,
b) modulating a function of a protein or polypeptide encoded by a detected nucleic acid by an antibody associated with said nucleic acid,
c) modulating a function of a protein or polypeptide encoded by a detected nucleic acid by a chemical compound such that said nucleic acid associates with said chemical compound,
d) assessing a function of a protein or polypeptide encoded by a detected nucleic acid wherein a cell is transformed by a nucleic acid comprising said detected nucleic acid, or
e) assessing a function of a protein or polypeptide encoded by a detected nucleic acid in a mammal harboring a transgene comprising said detected nucleic acid, or a combination of any two or more of said establishing procedures.
